(19) United States
(12) Patent Application Publication
(10) Pub. No.: US 2009/0325902 A1
(43) Pub. Date: Dec. 31, 2009

(54) HETEROCYCLIC UREA DERIVATIVES AND METHODS OF USE THEREOF

(75) Inventors: Brian Sherer, Waltham, MA (US); Fei Zhou, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/477,800

(22) Filed: Jun. 3, 2009

Related U.S. Application Data
(60) Provisional application No. 61/154,093, filed on Feb. 20, 2009.
(60) Provisional application No. 61/058,728, filed on Jun. 4, 2008.

(51) Int. Cl. A61K 31/695; C07D 401/14; A61K 31/444; A61P 31/04

(52) U.S. Cl. 514/63; 546/256; 514/333

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

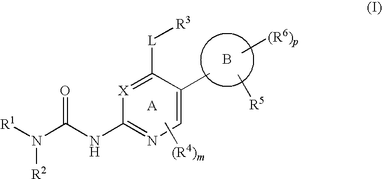

### RELATED APPLICATIONS

This application claims the benefit of U.S. application No. 61/058,728 filed on Jun. 4, 2008 and U.S. application No. 61/154,093 filed on Feb. 20, 2009. The entire teaching of U.S. 61/058,728 and U.S. 61/154,093 are incorporated herein by reference.

### FIELD OF THE INVENTION

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular, this invention relates to compounds useful for the treatment of bacterial infections in warm-blooded animals such as humans, more particularly to the use of these compounds in the manufacture of medicaments for use in the treatment of bacterial infections in warm-blooded animals such as humans.

### BACKGROUND OF THE INVENTION

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomerase IV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (U.S. Pat. No. 6,608,087). AstraZeneca has also published certain applications describing anti-bacterial compounds: WO2005/026149, WO2006/087544, WO2006/087548, WO2006/087543, WO2006/092599, WO2006/092608, and WO2007/071965.

### SUMMARY OF THE INVENTION

We have discovered a new class of compounds which are useful for inhibiting DNA gyrase and/or topoisomerase IV.

In one embodiment, according to the present invention there is provided a compound of formula (I):

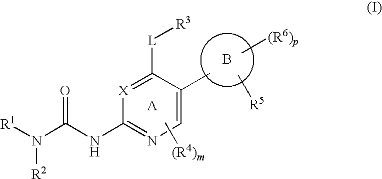

or a pharmaceutically acceptable salt thereof, wherein:

X is N, CH or CR⁴;

L is a C₁₋₆alkylene, —CH═CH—(C₁₋₄alkylene), or —C≡C—(C₁₋₄alkylene), wherein when L is —CH═CH—(C₁₋₄alkylene) or —C≡C—(C₁₋₄alkylene), the double or the triple bond is the point of attachment to Ring A;

R¹ is selected from C₁₋₆alkyl, C₂₋₆alkenyl, C₂₋₆alkynyl or C₃₋₆cycloalkyl; wherein R¹ may be optionally substituted on carbon by one or more R⁷;

R² is selected from hydrogen or C₁₋₆alkyl; wherein said C₁₋₆alkyl may be optionally substituted by one or more groups independently selected from halo, cyano, hydroxy, nitro and amino;

or R¹ and R² together with the nitrogen to which they are attached form a heterocyclyl; wherein said heterocyclyl may be optionally substituted on one or more carbon atoms with one or more R⁸; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R⁹;

R³ is hydrogen, a C₁₋₆alkyl, an (C₁₋₆alkyl)₃silyl, a C₃₋₁₄carbocyclyl or a heterocyclyl; wherein R³ may be optionally substituted on one or more carbon atoms by one or more R¹⁰; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹¹;

R⁴, for each occurrence, is independently selected from the group consisting of halo, nitro, cyano, hydroxy, amino, mercapto, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, and C₁₋₆alkylsulfanyl; wherein R⁴, for each occurrence, is independently optionally substituted on one or more carbon atoms with one or more R¹²;

R⁵ is hydrogen or a heterocyclyl; wherein the heterocyclyl may be optionally substituted on one or more carbon atoms with an ═O, ═S, or one or more R¹⁴; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁷;

R⁶, for each occurrence, is independently selected from the group consisting of halo, nitro, cyano, hydroxy, amino, mercapto, sulphamoyl, ═O, ═S, C₁₋₆alkyl, C₂₋₆alkenyl, C₂₋₆alkynyl, C₁₋₆alkoxy, N—(C₁₋₆alkyl)amino, N,N—(C₁₋₆alkyl)₂amino, C₁₋₆alkylS(O)ₐ— wherein a is 0, 1 or 2, N—(C₁₋₆alkyl)sulphamoyl, N,N—(C₁₋₆alkyl)₂sulphamoyl, C₁₋₆alkylsulphonylamino, C₃₋₁₄carbocyclyl and heterocyclyl; wherein R⁶, for each occurrence, is independently optionally substituted on one or more carbon atoms with one or more R¹⁶; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹³;

m is 0 or 1;

p is 0, 1, 2, or 3;

Ring B is C₃₋₁₄carbocyclyl or heterocyclyl; wherein if said heterocyclyl contains an —NH— moiety that nitrogen may be optionally substituted by a group selected from R¹⁵; and wherein if said heterocyclyl contains an ═N— or a —S— moiety that nitrogen may be optionally substituted by one oxo group and that sulfur may be optionally substituted by one or two oxo groups;

R⁷, R⁸, R¹⁰, R¹², R¹⁴ and R¹⁶ are substituents on carbon which, for each occurrence, are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ— wherein a is 0, 1 or 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, -L²-C₃₋₆carbocyclyl or -L²-heterocyclyl; wherein R⁷, R⁸, R¹⁰, R¹², R¹⁴ and R¹⁶ independently of each other may be optionally substituted on one or more carbon by one or more R¹⁹; and wherein if said heterocyclyl contains an —NH— moiety that nitrogen may be optionally substituted by a group selected from R²⁰; and wherein if said heterocyclyl contains an ═N— or a —S— moiety that nitrogen may be optionally substituted by one oxo group and that sulfur may be optionally substituted by one or two oxo groups;

L² is a direct bond, —O—, —N(R¹⁸)—, —C(O)—, —N(R¹⁸)C(O)—, —C(O)N(R¹⁸)—, —S(O)ₚ—, —SO₂N(R¹⁸)— or —N(R¹⁸)SO₂—; wherein R¹⁸, for each occurrence, is independently hydrogen or C₁₋₄alkyl and p is 0-2;

R⁹, R¹¹, R¹³, R¹⁵, R¹⁷, and R²⁰, for each occurrence, are independently selected from C₁₋₆alkyl, C₃₋₆cycloalkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁹, R¹¹, R¹³, R¹⁵, R¹⁷, and R²⁰ independently of each other may be optionally substituted on carbon by one or more R²³; and

R¹⁹ and R²³, for each occurrence, are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl.

In another embodiment, the invention provides pharmaceutical compositions comprising a compound represented by formula (I), or a pharmaceutically acceptable salt thereof, and a pharmaceutically acceptable excipient or carrier.

In another embodiment, the invention provides a method of inhibiting bacterial DNA gyrase and/or bacterial topoisomerase IV in a warm-blooded animal in need of such treatment, comprising administering to the animal an effective amount of a compound represented by formula (I), or a pharmaceutically acceptable salt thereof. In a particular embodiment, the warm-blooded animal is a human.

In another embodiment, the invention provides a method of producing an antibacterial effect in a warm-blooded animal in need of such treatment, comprising administering to the animal an effective amount of a compound represented by formula (I), or a pharmaceutically acceptable salt thereof. In a particular embodiment, the warm-blooded animal is a human.

In another embodiment, the invention provides a method of treating a bacterial infection in a warm-blooded animal in need thereof, comprising administering to the animal an effective amount of a compound represented by formula (I), or a pharmaceutically acceptable salt thereof. In a particular embodiment, the warm-blooded animal is a human. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides the use of a compound represented by formula (I), or a pharmaceutically acceptable salt thereof, for the manufacture of a medicament for use in the production of an antibacterial effect in a warm-blooded animal. In a particular embodiment, the warm-blooded animal is a human.

In another embodiment, the invention provides the use of a compound represented by formula (I), or a pharmaceutically acceptable salt thereof, for the manufacture of a medicament for use in inhibition of bacterial DNA gyrase and/or topoisomerase IV in a warm-blooded animal. In a particular embodiment, the warm-blooded animal is a human.

In another embodiment, the invention provides the use of a compound represented by formula (I), or a pharmaceutically acceptable salt thereof, for the manufacture of a medicament for use the treatment of a bacterial infection in a warm-blooded animal. In one embodiment, the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis and Vancomycin-Resistant Enterococci. In a particular embodiment, the warm-blooded animal is a human.

In another embodiment, the invention provides a compound represented by formula (I), or a pharmaceutically acceptable salt thereof, for use in production of an anti-bacterial effect in a warm-blooded animal.

In another embodiment, the invention provides a compound represented by formula (I), or a pharmaceutically acceptable salt thereof, for use in inhibition of bacterial DNA gyrase and/or topoisomerase IV in a warm-blooded animal.

In another embodiment, the invention provides a compound represented by formula (I), or a pharmaceutically acceptable salt thereof, for use in the treatment of a bacterial infection in a warm-blooded animal.

In another embodiment, the invention provides a compound represented by formula (I), or a pharmaceutically acceptable salt thereof, for use in the treatment of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.

### DETAILED DESCRIPTION OF THE INVENTION

In this specification the term alkyl includes both straight chained and branched saturated hydrocarbon groups. For example, “C₁₋₆alkyl” refers to an alkyl that has from 1 to 6 carbon atom and includes, for example, methyl, ethyl, propyl, isopropyl and t-butyl. However references to individual alkyl groups such as propyl are specific for the straight chain version only unless otherwise indicated (e.g., isopropyl). An analogous convention applies to other generic terms. Unless otherwise specified, when two or more alkyl groups are indicated by, for example, the term (C₁₋₆alkyl)₂ (such as in the term N,N—(C₁₋₆alkyl)₂amino), the alkyl groups can be the same or different.

As used herein, the term “alkylene” refers to a bivalent alkyl group which links two other groups. A “C₁₋₆alkylene” refers to an alkylene that has from 1 to 6 carbon atoms. An example of an alkylene is a methylene group.

As used herein, the term “alkene” refers to a straight chained or branched hydrocarbon that has one or more double bond. Examples of alkenes include ethenyl, 3-buten-1-yl, and the like. As used herein, the term “alkenylene” refers to a bivalent alkenyl group which links two other groups. A “C₁₋₆alkenylene” refers to an alkenylene that has from 1 to 6 carbon atoms. Examples of alkenylene include —CH═CH—, —CH₂CH═CHCH₂—, and the like.

As used herein, the term “alkynyl” refers to a straight chained or branched hydrocarbon that has one or more triple bond. Examples of alkynyl groups include ethynyl, 3-propyn-1-yl, and the like. As used herein, the term “alkynylene” refers to a bivalent alkynyl group which links two other groups. A “C₁₋₆alkynylene” refers to an alkynlene that has from 1 to 6 carbon atoms. Examples of alkynylene include —CH≡CH—, —CH₂CH≡CHCH₂—, and the like.

As used herein, the term “C₁₋₆haloalkyl” refers to an alkyl group that has from 1 to 6 carbon atoms in which one or more of the carbon atoms are substituted with a halo group. Representative haloalkyl groups include —CF₃, —CHF₂, —CCl₃, —CH₂CH₂Br, —CH₂CH(CH₂CH₂Br)CH₃, —CHICH₃, and the like.

As used herein, the term “halo” refers to fluoro, chloro, bromo, and iodo.

A “heterocyclyl” is a saturated, partially saturated or unsaturated, mono or bicyclic ring containing 4-14 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxide(s). In one embodiment of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. In a further aspect of the invention a “heterocyclyl” is unsaturated and aromatic. Examples and suitable values of the term “heterocyclyl” are morpholinyl, piperidyl, pyridinyl, pyranyl, pyrrolyl, pyrazolyl, isothiazolyl, indolyl, quinolinyl, thienyl, 1,3-benzodioxolyl, benzothiazolyl, thiadiazolyl, oxadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, 4,5-dihydro-oxazolyl, pyrimidinyl, pyrazinyl, pyridazinyl, isoxazolyl, thiazolyl, 1H-tetrazolyl, 1H-triazolyl, N-methylpyrrolyl, 4-pyridone, quinolin-4(1H)-one, pyridin-2(1H)-one, imidazo[1,2-a]pyridinyl, 1-isoquinolone, 2-pyrrolidone, 4-thiazolidone, quinoxalinyl, 5,6-dihydro[1,3]thiazolo[4,5-d]pyridazinyl, pyridine-N-oxide and quinoline-N-oxide. Suitable examples of “a nitrogen linked heterocyclyl” are morpholino, piperazin-1-yl, piperidin-1-yl and imidazol-1-yl. In a further aspect of the invention a “heterocyclyl” is unsaturated and aromatic. Examples and suitable values for an aromatic heterocycle include pyridinyl, pyrrolyl, pyrazolyl, isothiazolyl, indolyl, quinolinyl, thienyl, benzothiazolyl, thiadiazolyl, oxadiazolyl, imidazolyl, pyrimidinyl, pyrazinyl, pyridazinyl, isoxazolyl, thiazolyl, 1H-tetrazolyl, 1H-triazolyl, N-methylpyrrolyl, quinolin-4(1H)-one, pyridin-2(1H)-one, imidazo[1,2-a]pyridinyl, 1-isoquinolone, quinoxalinyl, pyridine-N-oxide and quinoline-N-oxide.

A “carbocyclyl” is a saturated, partially saturated or unsaturated, mono-, bi- or tricyclic carbon ring that contains 3-14 atoms; wherein a —CH₂— group can optionally be replaced by a —C(O)—. In one embodiment, “carbocyclyl” is a monocyclic ring containing 5 or 6 atoms or a bicyclic ring containing 9 or 10 atoms. Examples of carbocyclyls include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. The term carbocyclyl encompasses both cycloalkyl and aryl groups. In a particular embodiment, the carbocycle is a C₆₋₁₄aryl. A C₆₋₁₄aryl is an aromatic, mono-, bi- or tricyclic carbon ring that contains 6-14 atoms. Examples of aryl groups include phenyl and naphthyl.

As used herein, a “(C₁₋₆alkyl)₃silyl” is a silyl group that has three independently selected C₁₋₆alkyl groups, for example, trimethylsilyl and dimethyl-tertbutylsilyl.

An example of “C₁₋₆alkanoyloxy” is acetoxy. Examples of “C₁₋₆alkoxycarbonyl” are methoxycarbonyl, ethoxycarbonyl, n- and t-butoxycarbonyl. Examples of “C₁₋₆alkoxycarbonylamino” are methoxycarbonylamino, ethoxycarbonylamino, n- and t-butoxycarbonylamino. Examples of “C₁₋₆alkoxy” are methoxy, ethoxy and propoxy. Examples of “C₁₋₆alkanoylamino” are formamido, acetamido and propionylamino. Examples of “C₁₋₆alkylS(O)ₐ wherein a is 0, 1, or 2” are methylthio, ethylthio, methylsulphinyl, ethylsulphinyl, mesyl and ethylsulphonyl. Examples of “C₁₋₆alkanoyl” are propionyl and acetyl. Examples of “N—(C₁₋₆alkyl)amino” are methylamino and ethylamino. Examples of “N,N—(C₁₋₆alkyl)₂amino” are di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “N—(C₁₋₆alkoxy)carbamoyl” are methoxyaminocarbonyl and isopropoxyaminocarbonyl. Examples of “N—(C₁₋₆alkyl)-N—(C₁₋₆alkoxy)carbamoyl” are N-methyl-N-methoxyaminocarbonyl and N-methyl-N-ethoxyaminocarbonyl. Examples of “C₃₋₆cycloalkyl” are cyclopropyl, cyclobutyl, cyclopropyl and cyclohexyl. Examples of “C₁₋₆alkylsulphonylamino” are methylsulphonylamino, isopropylsulphonylamino and t-butylsulphonylamino. Examples of “C₁₋₆alkylsulphonylaminocarbonyl” are methylsulphonylaminocarbonyl, isopropylsulphonylaminocarbonyl and t-butylsulphonylaminocarbonyl. Examples of “C₁₋₆alkylsulphonyl” are methylsulphonyl, isopropylsulphonyl and t-butylsulphonyl.

The term “formula (I)”, unless otherwise specified, refers to all embodiments of formula (I) including but not limited to formula (Ia), formula (Ib), and formula (Ic).

A compound of formula (I) may form stable acid or basic salts, and in such cases administration of a compound as a salt may be appropriate, and pharmaceutically acceptable salts may be made by conventional methods such as those described below.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I), or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that certain compounds of formula (I) contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase and/or topoisomerase IV, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase and/or topoisomerase IV by the standard tests described hereinafter.

By way of clarity, compounds of the invention included all isotopes of the atoms present in formula (I) and any of the examples or embodiments disclosed herein. For example, H (or hydrogen) represents any isotopic form of hydrogen including ¹H, ²H (D), and 3H (T); C represents any isotopic form of carbon including ¹²C, ¹³C, and ¹⁴C; O represents any isotopic form of oxygen including ¹⁶O, ¹⁷O and ¹⁸O; N represents any isotopic form of nitrogen including ¹³N, ¹⁴N and ¹⁵N; P represents any isotopic form of phosphorous including ³¹P and ³²P; S represents any isotopic form of sulfur including ³²S and ³⁵S; F represents any isotopic form of fluorine including ¹⁹F and ¹⁸F; Cl represents any isotopic form of chlorine including ³⁵Cl, ³⁷Cl and ³⁶Cl; and the like. In a preferred embodiment, compounds represented by formula (I) comprises isomers of the atoms therein in their naturally occurring abundance. However, in certain instances, it is desirable to enrich one or more atom in a particular isotope which would normally be present in less abundance. For example, ¹H would normally be present in greater than 99.98% abundance; however, a compound of the invention can be enriched in ²H or ³H at one or more positions where H is present. In particular embodiments of the compounds of formula (I), when, for example, hydrogen is enriched in the deuterium isotope, the symbol “D” is used to represent the enrichment in deuterium. In one embodiment, when a compound of the invention is enriched in a radioactive isotope, for example ³H and ¹⁴C, they may be useful in drug and/or substrate tissue distribution assays. It is to be understood that the invention encompasses all such isotopic forms which inhibit DNA gyrase and/or topoisomerase IV.

It is also to be understood that certain compounds of the formula (I), and salts thereof can exist in solvated as well as unsolvated forms such as, for example, hydrated forms. It is to be understood that the invention encompasses all such solvated forms which inhibit DNA gyrase and/or topoisomerase IV.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

In one embodiment the invention provides compounds represented by formula (I) wherein X is CH.

In another embodiment the invention provides compounds represented by formula (I) wherein X is N.

In another embodiment the invention provides compounds represented by formula (I) wherein X is CR⁴ and R⁴ is fluoro, chloro, bromo, iodo, a C₁₋₄alkyl, or a C₁₋₄alkoxy.

In another embodiment the invention provides compounds represented by formula (I) wherein L is a C₂₋₆alkynylene, for example —C≡C—. In a particular embodiment, L is —C≡C—(C₁₋₄alkylene)

In another embodiment the invention provides compounds represented by formula (I) wherein L is a C₂₋₆alkenylene. In a particular embodiment, L is —CH═CH—(C₁₋₄alkylene).

In another embodiment the invention provides compounds represented by formula (I) wherein L is a C₁₋₆alkylene.

In another embodiment the invention provides compounds represented by formula (I) wherein ring B is a 5- or 6-membered heteroaryl, and wherein if said heteroaryl contains an —NH— moiety that nitrogen may be optionally substituted by a group selected from R¹⁵; and wherein if said heteroaryl contains an ═N— or a —S— moiety that nitrogen may be optionally substituted by one oxo group and that sulfur may be optionally substituted by one or two oxo groups.

In another embodiment the invention provides compounds represented by formula (I) wherein ring B is pyridinyl, pyrazinyl, pyrimidinyl or thiazolyl; and wherein each ═N— of pyridinyl, pyrazinyl, pyrimidinyl, or thiazolyl may be independently optionally substituted with one oxo group; and wherein the —S— moiety of the thiazolyl may be optionally by one or two oxo groups.

In another embodiment the invention provides compounds represented by formula (I) wherein ring B is a bicyclic heterocyclyl; and wherein if said heterocyclyl contains an —NH— moiety that nitrogen may be optionally substituted by a group selected from R¹⁵; and wherein if said heterocyclyl contains an ═N— or a —S— moiety that nitrogen may be optionally substituted by one oxo group and that sulfur may be optionally substituted by one or two oxo groups.

In another embodiment the invention provides compounds represented by formula (I) wherein ring B is a quinoxalinyl or 5,6-dihydro[1,3]thiazolo[4,5-d]pyridazine-4,7-dione; and wherein each —NH— moiety of 5,6-dihydro[1,3]thiazolo[4,5-d]pyridazine-4,7-dione may be independently optionally substituted by a group selected from R¹⁵; and wherein each ═N— of quinoxalinyl or 5,6-dihydro[1,3]thiazolo[4,5-d]pyridazine-4,7-dione may be independently optionally substituted with one oxo group; and wherein wherein the —S— moiety of the 5,6-dihydro[1,3]thiazolo[4,5-d]pyridazine-4,7-dione may be optionally by one or two oxo groups.

In another embodiment the invention provides compounds represented by formula (I) wherein ring B is pyridinyl; and wherein ═N— may be optionally substituted with one oxo group. In one embodiment, ring B is and unsubstituted pyridinyl.

In another embodiment the invention provides compounds represented by formula (I) wherein ring B is a C₃₋₁₄carbocyclyl, for example a phenyl.

In another embodiment the invention provides compounds represented by formula (I) wherein R¹ is a C₁₋₆alkyl. For example, R¹ is methyl, ethyl, propyl, isopropyl, butyl, isobutyl, sec-butyl, and tert-butyl. In a particular embodiment, R¹ is ethyl.

In another embodiment the invention provides compounds represented by formula (I) wherein R² is hydrogen.

In another embodiment the invention provides compounds represented by formula (I) wherein R² is a C₁₋₆alkyl. For example, R² is methyl, ethyl, propyl, isopropyl, butyl, isobutyl, sec-butyl, and tert-butyl.

In another embodiment the invention provides compounds represented by formula (I) wherein R³ is a 5-membered heteroaryl; and wherein the heteroaryl may be optionally substituted on one or more carbon atoms by one or more R¹⁰; and wherein if said heteroaryl contains an ═N— or a —S— moiety that nitrogen may be optionally substituted by one oxo group and that sulfur may be optionally substituted by one or two oxo groups; and wherein if said heteroaryl contains an —NH— moiety that nitrogen may be optionally substituted by a group selected from R¹¹. In one aspect of this embodiment, R¹⁰ is selected from the group consisting of methyl, phenyl, trifluoromethyl, and pyridinyl. In another aspect of this embodiment, R¹¹ is methyl.

In another embodiment the invention provides compounds represented by formula (I) wherein R³ is a thiazolyl; and wherein the thiazolyl may be optionally substituted on carbon by one or more R¹⁰; and wherein the ═N— of the thiazolyl may be optionally substituted by one oxo group; and wherein the —S— of the thiazolyl may be optionally substituted by one or two oxo groups. In one aspect of this embodiment, R¹⁰ is selected from the group consisting of methyl, phenyl, trifluoromethyl, and pyridinyl. In another aspect of this embodiment, R¹¹ is methyl.

In another embodiment the invention provides compounds represented by formula (I) wherein R³ is a 1,3,4-oxadiazolyl; and wherein the 1,3,4-oxadiazolyl may be optionally substituted on one or more carbon by one or more R¹⁰; and wherein each ═N— of the 1,3,4-oxadiazolyl may be independently optionally substituted by one oxo group. In one aspect of this embodiment, R¹⁰ is selected from the group consisting of methyl, phenyl, trifluoromethyl, and pyridinyl. In another aspect of this embodiment, R¹¹ is methyl.

In another embodiment the invention provides compounds represented by formula (I) wherein R³ is a 1H-pyrazolyl; and wherein the 1H-pyrazolyl may be optionally substituted on one or more carbon by one or more R¹⁰; and wherein the ═N— of the 1H-pyrazolyl may be optionally substituted by one oxo group; and wherein the —NH— moiety of the 1H-pyrazolyl may be optionally substituted by a group selected from R¹¹. In one aspect of this embodiment, R¹⁰ is selected from the group consisting of methyl, phenyl, trifluoromethyl, and pyridinyl. In another aspect of this embodiment, R¹¹ is methyl.

In another embodiment the invention provides compounds represented by formula (I) wherein R³ is 1,3-benzothiazolyl; and wherein the 1,3-benzothiazolyl may be optionally substituted on one or more carbon by one or more R¹⁰; and wherein the ═N— of the 1,3-benzothiazolyl may be optionally substituted by one oxo group; and wherein the —S— of the 1,3-benzothiazolyl may be optionally substituted by one or two oxo groups. In one aspect of this embodiment, R¹⁰ is selected from the group consisting of methyl, phenyl, trifluoromethyl, and pyridinyl. In another aspect of this embodiment, R¹¹ is methyl.

In another embodiment, R³ is a (C₁₋₆alkyl)₃silyl which is optionally substituted on one or more carbon atoms with one or more independently selected R¹⁰. In one embodiment, R³ is trimethylsilyl.

In another embodiment, R³ is a C₁₋₆alkyl which is optionally substituted on one or more carbon atoms with one or more independently selected R¹⁰. In one embodiment, R¹⁰, for each occurrence is independently selected from a C₁₋₆alkoxy, —O—C₃₋₆carbocycle, and —O— heterocyclyl. In one embodiment, R³ is methoxymethyl. In another embodiment, R³ is tetrahydro-2H-pyran-2-yloxy.

In another embodiment the invention provides compounds represented by formula (I) wherein R³ is 4-trifluoromethy-thiazol-2-yl, 4-(pyridin-2-yl)-thiazol-2-yl, 4-phenyl-thiazol-2-yl, 1,3-benzothiazol-2-yl, 2-(pyridin-4-yl)-1,3,4-oxadiazol-5-yl, 1-methyl-1H-pyrazol-5-yl, 1-methyl-1H-pyrazol-4-yl, 2-methyl-1,3,4-oxadiazol-5-yl, or 4-(pyridin-4-yl)-thiazol-2-yl.

In another embodiment the invention provides compounds represented by formula (I) wherein R³ is pyridinyl; and wherein ═N— may be optionally substituted with one oxo group. In one embodiment, R³ is an unsubstituted pyridinyl.

In another embodiment the invention provides compounds represented by formula (I) wherein R³ is a C₆₋₁₄aryl which may be optionally substituted on one or more carbon atoms with one or more R¹⁰.

In another embodiment the invention provides compounds represented by formula (I) wherein R⁵ is a five membered aromatic heterocyclyl; and wherein the heterocyclyl may be optionally substituted on one or more carbon atoms with one or more R¹⁴; and wherein if said heterocyclyl contains an ═N— or a —S— moiety that nitrogen may be optionally substituted by one oxo group and that sulfur may be optionally substituted by one or two oxo groups; and wherein if said heterocyclyl contains an —NH— moiety that nitrogen may be optionally substituted by a group selected from R¹⁷. In one aspect of this embodiment, R¹⁴ is selected from the group consisting of C₁₋₄alkyl or hydroxy. In another aspect of this embodiment, R¹⁷ is a C₁₋₄alkyl.

In another embodiment the invention provides compounds represented by formula (I) wherein R⁵ is selected from the group consisting of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl, wherein the 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, morpholinyl, 4,5-dihydro-oxazolyl, and 1H-1,2,4-triazolyl may be optionally substituted on one or more carbon atoms with one or more R¹⁴; and wherein the ═N— moiety of 1,3,4-oxadiazolyl, 1,3,4-thiadiazolyl, 1H-tetrazolyl, 1,2,4-oxadiazolyl, 1H-pyrazolyl, 3H-1,2,3,5-oxathiadiazolyl, 1H-imidazolyl, 4,5-dihydro-oxazolyl, and 1H-1,2,4-triazolyl may be optionally substituted with one oxo group and the —S— moiety of 1,3,4-thiadiazolyl or 3H-1,2,3,5-oxathiadiazolyl may be optionally substituted by one or two oxo groups; and wherein the —NH— moiety of the 1H-tetrazolyl, 1H-pyrazolyl, 3H-1,2,3,5-oxathiadiazolyl, 1H-imidazolyl, morpholinyl, or the 1H-1,2,4-triazolyl may be optionally substituted by a group selected from R¹⁷. In one aspect of this embodiment, R¹⁴ is selected from the group consisting of C₁₋₄alkyl or hydroxy. In another aspect of this embodiment, R¹⁷ is a C₁₋₄alkyl.

In another embodiment the invention provides compounds represented by formula (I) wherein R⁵is a group represented by the following formula

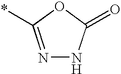

wherein “*” represents the point of attachment to ring B.

In another embodiment the invention provides compounds represented by formula (I) wherein m is 0.

In another embodiment the invention provides compounds represented by formula (I) wherein m is 0 and X is CH.

In another embodiment the invention provides compounds represented by formula (I) wherein m is 0 and X is N.

In another embodiment the invention provides compounds represented by formula (I) wherein m is 1.

In another embodiment the invention provides compounds represented by formula (I) wherein p is 0.

In another embodiment the invention provides compounds represented by formula (I) wherein p is 0 and R⁵ is hydrogen. In one aspect of this embodiment, ring B is pyridine or quinoxalinyl.

In another embodiment the invention provides compounds represented by formula (I) wherein p is 1. In one aspect of this embodiment, R⁶ is cyano, bromo, methylsulfonyl, sulphamoyl, or butyloxy.

In another embodiment the invention provides compounds represented by formula (I) wherein p is 1 and R is hydrogen. In one aspect of this embodiment, R⁶ is cyano, bromo, methylsulfonyl, sulphamoyl, or butyloxy.

In another embodiment the invention provides compounds represented by formula (I) wherein p is 2. In one aspect of this embodiment, R⁶, for each occurrence, is independently selected from cyano, bromo, methylsulfonyl, sulphamoyl, and butyloxy.

In another embodiment the invention provides compounds represented by formula (I) wherein p is 3. In one aspect of this embodiment, R⁶, for each occurrence, is independently selected from cyano, bromo, methylsulfonyl, sulphamoyl, and butyloxy.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a thiazolyl; wherein the thiazolyl may be optionally substituted on carbon by one or more R¹⁰;

R⁵ is selected from the group consisting of 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl; wherein the 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl may be optionally substituted on one or more carbon atoms with one or more R¹⁴; and wherein the —NH— moiety of the 1H-tetrazolyl, 1H-pyrazolyl, 1H-imidazolyl, morpholinyl, or the 1H-1,2,4-triazolyl may be optionally substituted by methyl;

R¹⁰ is trifluoromethyl pyridinyl, phenyl, 1-methyl-1H-pyrazolyl;

m is 0; and

p is 0.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a pyridinyl;

R⁵ is selected from the group consisting of 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl; wherein the 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl may be optionally substituted on one or more carbon atoms with one or more R¹⁴; and wherein the —NH— moiety of the 1H-tetrazolyl, 1H-pyrazolyl, 1H-imidazolyl, morpholinyl, or the 1H-1,2,4-triazolyl may be optionally substituted by methyl;

m is 0; and

p is 0.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a pyridinyl;

R⁵ is selected from the group consisting of 5-hydroxy-1,3,4-oxadiazol-2-yl;

m is 0; and

p is 0.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a hydrogen;

R⁵ is selected from the group consisting of 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl; wherein the 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl may be optionally substituted on one or more carbon atoms with one or more R¹⁴; and wherein the —NH— moiety of the 1H-tetrazolyl, 1H-pyrazolyl, 1H-imidazolyl, morpholinyl, or the 1H-1,2,4-triazolyl may be optionally substituted by methyl;

m is 0; and

p is 0.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a hydrogen;

R⁵ is selected from the group consisting of 5-hydroxy-1,3,4-oxadiazol-2-yl;

m is 0; and

p is 0.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a trimethylsilyl;

R⁵ is selected from the group consisting of 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl; wherein the 1,3,4-oxadiazolyl, 1H-tetrazolyl, 1,3,4-thiadiazolyl, 1H-1,2,4-triazolyl, 1,2,4-oxadiazolyl, 4,5-dihydro-oxazolyl, 1H-pyrazolyl, 2-oxo-3H-1,2,3,5-oxathiadiazolyl, 1H-imidazolyl, and morpholinyl may be optionally substituted on one or more carbon atoms with one or more R¹⁴; and wherein the —NH— moiety of the 1H-tetrazolyl, 1H-pyrazolyl, 1H-imidazolyl, morpholinyl, or the 1H-1,2,4-triazolyl may be optionally substituted by methyl;

m is 0; and

p is 0.

In a particular embodiment, the present invention provides compounds having a structural formula (I) as recited above wherein:

X is CH;

L is —C≡C—;

Ring B is pyridinyl;

R¹ is C₁₋₄alkyl;

R² is hydrogen;

R³ is a trimethylsilyl;

R⁵ is selected from the group consisting of 5-hydroxy-1,3,4-oxadiazol-2-yl;

m is 0; and

p is 0.

In a particular embodiment, the present invention provides compounds having a structural formula (I) as recited above wherein:

X is CH;

L is —C≡C—;

Ring B is pyridinyl;

p is 1;

R¹ is C₁₋₄alkyl;

R² is hydrogen;

R³ is a thiazolyl; wherein the thiazolyl may be optionally substituted on carbon by one or more R¹⁰;

R⁵ is hydrogen;

R⁶ is sulfamoyl, mesyl, cyano, or halo;

R¹⁰ is trifluoromethyl pyridinyl, phenyl, 1-methyl-1H-pyrazolyl; and

m is 0.

In a particular embodiment, the present invention provides compounds having a structural formula (I) as recited above wherein:

X is CH;

L is —C≡C—;

Ring B is pyridinyl, quinoxalinyl or 5,6-dihydro[1,3]thiazolo[4,5-d]pyridazine-4,7-dione;

R¹ is C₁₋₄alkyl;

R² is hydrogen;

R³ is a thiazolyl; wherein the thiazolyl may be optionally substituted on carbon by one or more R¹⁰;

R⁵ is hydrogen;

R¹⁰ is trifluoromethyl pyridinyl, phenyl, 1-methyl-1H-pyrazolyl;

m is 0; and

p is 0.

Particular compounds of the invention are the compounds of the Examples, and pharmaceutically acceptable salts thereof, each of which provides a further independent aspect of the invention.

In another embodiment, the invention provides pharmaceutical compositions comprising a pharmaceutically acceptable excipient or carrier and a compound represented by formula (I), or a pharmaceutically acceptable salt thereof.

In a further aspect the present invention provides a process for preparing a compound of formula (I), or a pharmaceutically-acceptable salt thereof, wherein variable groups in the schemes below are as defined in formula (I) unless otherwise specified. In general, the compounds of the invention can be prepared by a palladium catalyzed Suzuki coupling reaction of a boronic ester derivative (i) or (v) and a halo or triflate derivative (ii) or (iv), as shown in Schemes I and II, followed by a Heck reaction or Sonogashira reaction to add a alkenyl linker or alkynyl linker (respectively) and an R³ group (see Scheme III, formulae (Ia) and (Ib)). Typically, the Suzuki coupling reaction is heated and is carried out in the presence of a base such as Cs₂CO₃. Formula (Ia) or (Ib) can be hydrogenated using a hydrogen source, such as hydrogen gas, and a metal catalyst, such as platinum, palladium, rhodium, ruthenium, or nickel catalyst, to form compounds of the invention which have an alkylene linked R³ group (Scheme III, formula (Ic)). Although Scheme III shows the Heck or Sonogahira reaction to add the akenylene and alkynylene linker after the Suzuki coupling reaction, the reactions could be preformed in the alternative order.

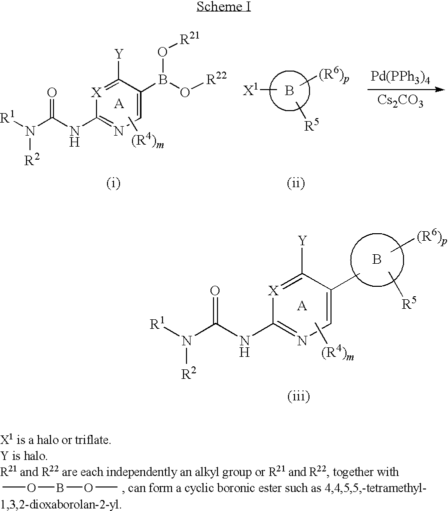

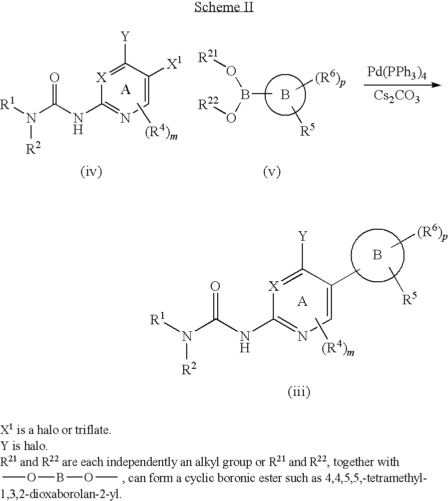

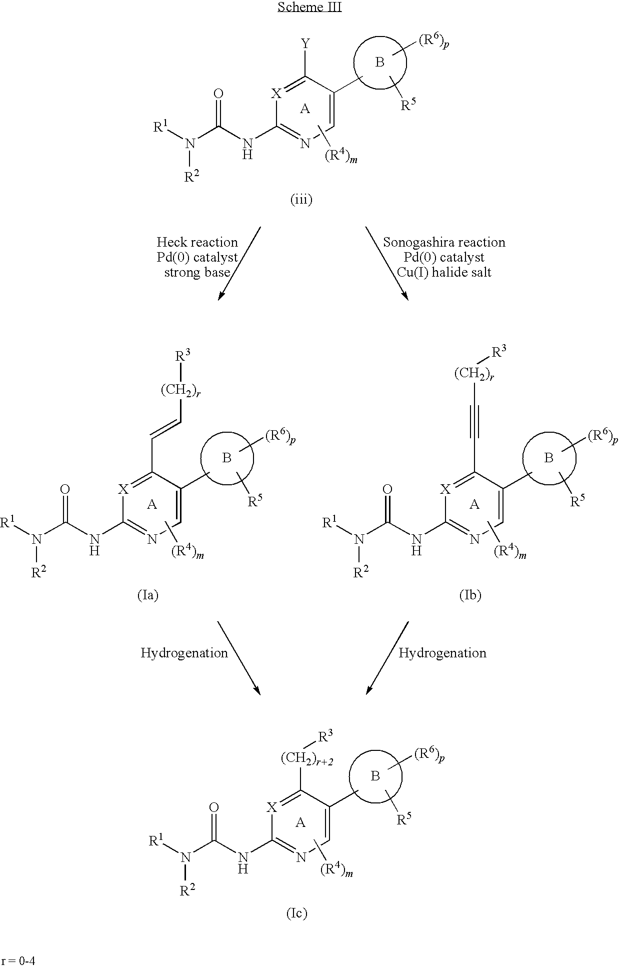

Boronic ester derivatives can be prepared by heating a halo derivative with a diboron compound such as 4,4,4′,4′,5,5,5′,5′-octamethyl-2,2′-bi(1,3,2-dioxaborolane) in the presence of 1,1′-bis(diphenylphosphino)ferrocene-palladium dichloride in an organic solvent.

The urea portion of the compounds of the invention can be prepared from an isocyanate derivative either before (as shown in Schemes I and II) or after the Suzuki coupling reaction or before or after addition of -L-R³ from an amine derivative. If the Suzuki coupling reaction or addition of -L-R³is preformed before formation of the urea, the amine is protected with an amine protecting group. When forming the urea derivative, the isocyanate derivative (vii) is typically combined with the amine derivative (vi) in an organic solvent and heated, as shown in Scheme IV. The solvent can be aqueous, organic or a mixture of an aqueous miscible organic solvent and water.

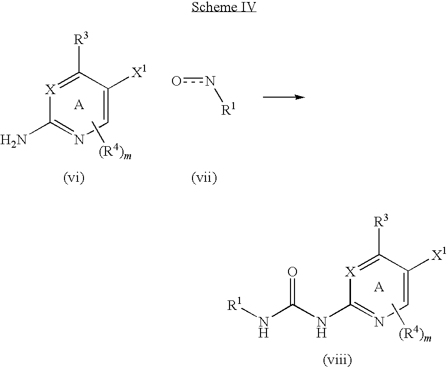

In general, when R⁵ is a heterocyclyl, it can be added by a Suzuki coupling reaction analogous to that shown in Schemes I and II. R can be coupled to ring B either before or after ring B is coupled to ring A or before or after addition of -L-R³.

Alternatively, when R⁵ is a heterocyclyl, it can be prepared from an ester derivative either before or after coupling of ring B to ring A. For example, when R⁵ is a thiazolyl group, an ester derivative (ix) can be converted to an amide (x) by treating it with a solution of ammonia in an alcohol. The amide derivative (x) can then be converted to a thioamide (xi) by treating the amide with Lawessons reagent. The thioamide (xi) is then heated with an α-halo-ketone or an α-halo-aldehyde (xii) followed by treatment with an acid such as trifluoroacetic acid to form the thiazole (xii) (see Scheme V). Although the thiazole ring is prepared before the Suzuki coupling reaction to attach ring A to ring B in Scheme V, it could also be prepared after the coupling reaction of the ester derivative. Likewise, an R⁵ thiazole ring could be prepared either before or after addition of R³-L- to ring A.

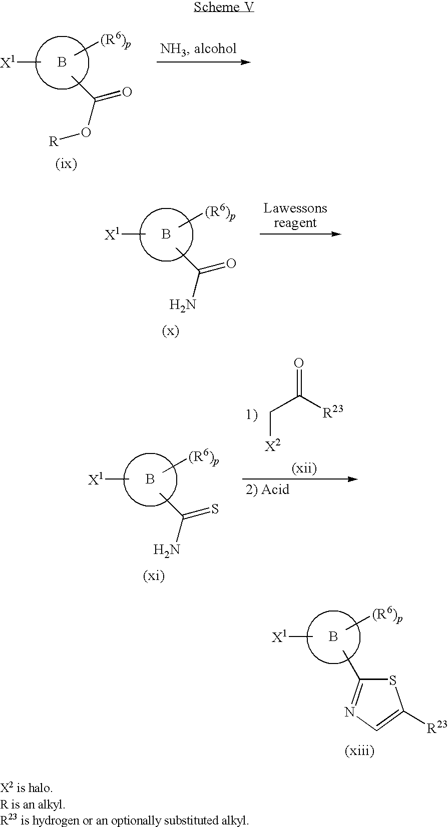

When R⁵ is tetrazolyl, it can be prepared by heating a cyano derivative with sodium azide and ammonium chloride in a solvent as shown in Scheme VI. R⁵ tetrazolyl groups can be prepared by the reaction shown in Scheme VI either before or after coupling of ring B to ring A or before or after addition of R³-L-.

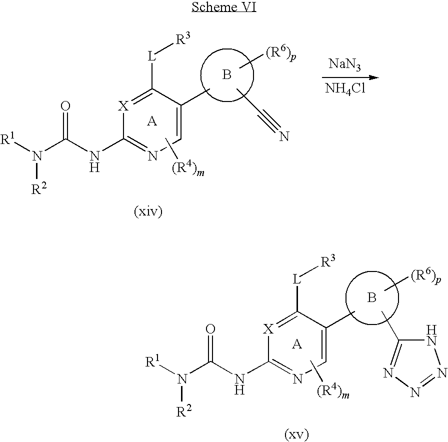

When R⁵ is a 1,3,4-oxadiazolyl group, it can be prepared from an ester derivative (xvi) by treating the ester with a base in to form a carboxylic acid (xvii). The carboxylic acid (xvii) is then coupled to a hydrazide derivative (xviii) in the presence of the amide coupling reagent HATU to form a dihydrazide derivative (xix). The dihydrazide (xix) is then treated with triphenyl phosphine in an aprotic organic solvent in the presence of an excess amount of an aprotic base to form a compound of the invention in which the R⁵ group is 1,3,4-oxadiazolyl (xx) as shown in Scheme VII. An R⁵ 1,3,4-oxadiazolyl group can be prepared by the reaction shown in Scheme VII either before or after coupling of ring B to ring A or before or after addition of R³-L-.

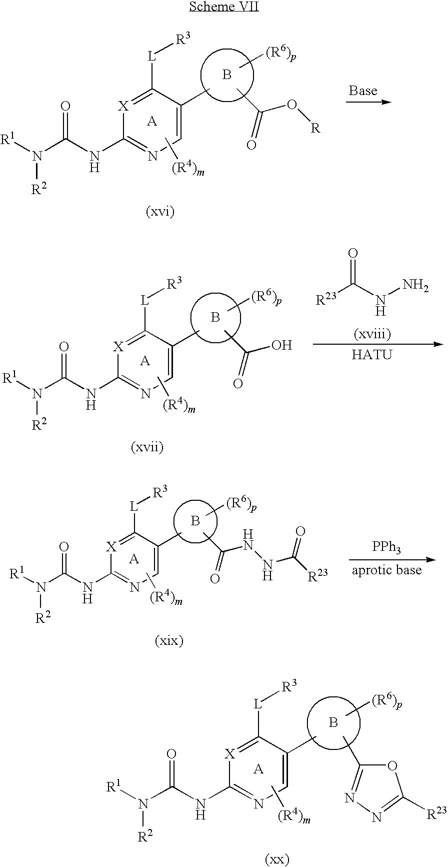

When R⁵ is a 1,3,4-thiadiazolyl group, it can be prepared from a dihydrazide derivative (xix) (see Scheme VII for preparation of dihydrazide derivatives). The dihydrazide derivative (xix) is heated with phosphorous pentasulfide and hexamethyldisiloxane in an organic solvent to form a compound of the invention having an R⁵ 1,3,4-thiadiazolyl group (xxi) as shown in Scheme VIII. An R⁵ 1,3,4-thiadiazolyl group can be prepared by the reaction shown in Scheme VIII either before or after coupling of ring B to ring A or before or after addition of -L-R³.

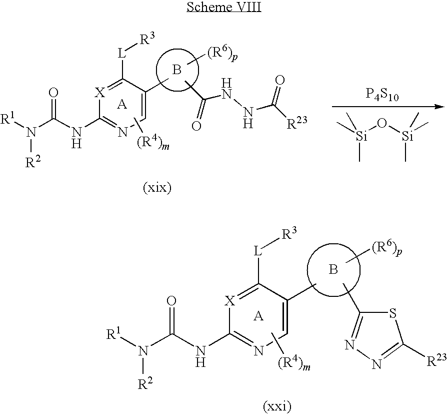

When R⁵is a 2-oxo-1,3,4-oxadiazolyl or a 2-thioxo-1,3,4-oxadiazolyl group, it can be prepared from a carboxylic acid (xvii) (see Scheme VII for preparation of the carboxylic acid derivative). The carboxylic acid derivative (xvii) is heated with hydrazine hydrate in an alcohol to form a hydrazide derivative (xxii). The hydrazide derivative (xxii) is then reacted with carbonyl diimidazole or di(1-H-imidazol-2-yl)methanethione (xxiii) in the presence of an aprotic base in an aprotic solvent to form a compound of the invention which has an R⁵ 2-oxo-1,3,4-oxadiazolyl or a 2-thioxo-1,3,4-oxadiazolyl group (xxiv) as shown in Scheme IX. An R⁵ 2-oxo-1,3,4-oxadiazolyl or a 2-thioxo-1,3,4-oxadiazolyl group can be prepared by the reaction shown in Scheme IX either before or after coupling of ring B to ring A or before or after addition of -L-R³.

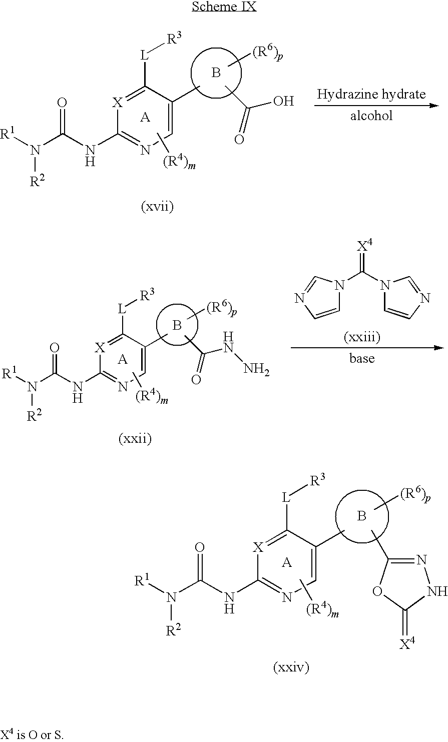

When R⁵ is a 1,2,4-triazolyl group, it can be prepared from an amide derivative (xxv) by heating it in 1-(N,N-dimethylamino)-1,1-dimethoxy-ethane (xxvi) to form compound (xxvii). Compound (xxvii) is then heated with acetohydrazide in acetic acid to form a compound of the invention that has an R⁵ 1,2,4-triazolyl group (xxviii) as shown in Scheme X. An R⁵ 1,2,4-triazolyl group can be prepared by the reaction shown in Scheme X either before or after coupling of ring B to ring A or before or after addition of -L-R³.

When R⁵ is a 1,2,4-oxadiazolyl group, it can be prepared from (xxvii) by heating (xxvii) with hydroxyl amine hydrochloride in a solution of sodium hydroxide in 70% acetic acid in dioxane to form a compound of the invention in which R⁵ is a 1,2,4-oxadiazolyl group (xxix) as shown in Scheme X. An R⁵ 1,2,4-oxadiazolyl group can be prepared by the reaction shown in Scheme X either before or after coupling of ring B to ring A or before or after addition of -L-R³.

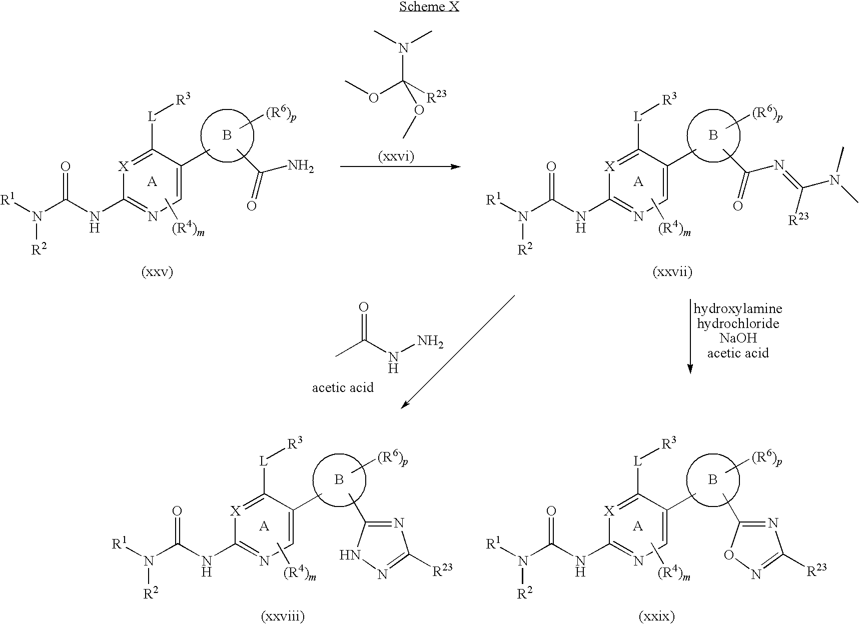

When R⁵ is an imidazolyl group, it can be prepared from a cyano derivative (xiv) by stirring the cyano derivative (xiv) at room temperature in a solution of sodium methoxide in methanol for several hours. 1,1-Dimethoxy-2-aminoethane (xxx) is then added to the solution and it is heated to give a compound of the invention in which R⁵is an imidazolyl group (xxxi) as shown in Scheme XI. An R⁵ imidazolyl group can be prepared by the reaction shown in Scheme XI either before or after coupling of ring B to ring A or before or after addition of -L-R³.

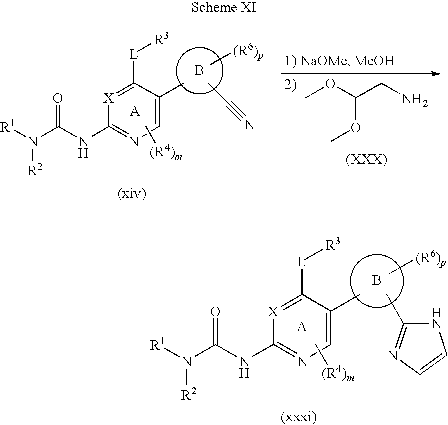

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon, or for example, an allyl group which may be removed, for example, by use of a palladium catalyst such as palladium acetate.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

E. coli GyrB ATPase Inhibition Activity: Compounds can be tested for inhibition of E. coli GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1 mM 1,4-Dithio-DL-threitol, 200 nM bovine serum albumin, 1.6 μg/ml sheared salmon sperm DNA, 400 pM E. coli GyrA, 400 pM E. coli GyrB, 250 μM ATP, and compound in dimethylsulfoxide. Reactions can be quenched with 30 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values are calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

E. coli Topoisomerase IV ATPase Inhibition Activity: Compounds can be tested for inhibition of E. coli topoisomerase IV ATPase activity as described above for E. coli GyrB except the 30 μl reactions contained the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 500 pM E. coli ParC, 500 pM E. coli ParE, 160 μM ATP, and compound in dimethylsulfoxide. An IC₅₀ measurement of compound potency for each compound can be determined from reactions performed in the presence of 10 different compound concentrations.

The compound in Example 1 was tested in an assay substantially similar to the assays described above for measuring the inhibition of E. coli GyrB ATPase and E. coli Topoisomerase IV ATPase and had an IC₅₀ values of <200 μM in both assays.

S. aureus GyrB ATPase Inhibition Activity: Compounds may be tested for inhibition of S. aureus GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 30 μl reactions containing: 50 mM Hepes buffer pH 7.5, 75 mM ammonium acetate, 8.0 mM magnesium chloride, 0.5 mM ethylenediaminetetraacetic acid, 5% glycerol, 1.0 mM 1,4-Dithio-DL-threitol, 200 nM bovine serum albumin, 1.0 μg/ml sheared salmon sperm DNA, 250 pM E. coli GyrA, 250 pM S. aureus GyrB, 250 μM ATP, and compound in dimethylsulfoxide. Reactions can be quenched with 30 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values can be calculated using dimethylsulfoxide (2%)-containing reactions as 0% inhibition and EDTA-containing (2.4 μM) reactions as 100% inhibition controls. An IC₅₀ measurement of compound potency for each compound can be determined from reactions performed in the presence of 10 different compound concentrations.

The compound in Example 1 was tested in an assay substantially similar to the assay described above for measuring the inhibition of S. aureus GyrB ATPase and was found to have a percent inhibition of S. aureus GyrB ATPase at a compound concentration of 1 μM of 102%.

### Bacterial Susceptibility Testing Methods

Compounds may be tested for antimicrobial activity by susceptibility testing in liquid media. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension can be a 0.5 McFarland and a further 1 in 10 dilution can be made into the same liquid medium to prepare the final organism suspension in 100 μL. Plates can be incubated under appropriate conditions at 37° C. for 24 hrs prior to reading. The Minimum Inhibitory Concentration (MIC) may be determined as the lowest drug concentration able to reduce growth by 80% or more.

In an assay comparable to the above, Example 1 had an MIC of 0.2 μM against Streptococcus pneumoniae.

According to a further feature of the invention there is provided a compound of the formula (I), or a pharmaceutically-acceptable salt thereof, for use in a method of treatment of the human or animal body by therapy.

In one embodiment, the invention provides a method of treating a bacterial infection in an animal, such as a human, comprising administering to the animal or human an effective amount of a compound of any one of formulas (I), or a pharmaceutically acceptable salt thereof.

We have found that compounds of the present invention inhibit bacterial DNA gyrase and/or topoisomerase IV and are therefore of interest for their antibacterial effects. In one aspect of the invention the compounds of the invention inhibit bacterial DNA gyrase and are therefore of interest for their antibacterial effects. In one aspect of the invention, the compounds of the invention inhibit topoisomerase IV and are therefore of interest for their antibacterial effects. In one aspect of the invention, the compounds of the invention inhibit both DNA gyrase and topoisomerase IV and are therefore of interest for their antibacterial effects. Thus, the compounds of the invention are useful in treating or preventing bacterial infections.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficile. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptococcus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis. In one aspect of the invention an “infection” or “bacterial infection” refers to an infection caused by Mycobacterium tuberculosis In one aspect of the invention an “infection” or “bacterial infection” refers to an infection caused by Clostridium perfringens. In one aspect of the invention an “infection” or “bacterial infection” refers to an infection caused by Klebsiella oxytoca. In one aspect of the invention an “infection” or “bacterial infection” refers to an infection caused by Neisseria miningitidis. In one aspect of the invention an “infection” or “bacterial infection” refers to an infection caused by Fusobacterium spp. In one aspect of the invention an “infection” or “bacterial infection” refers to an infection caused by Peptococcus spp. In one aspect of the invention an “infection” or “bacterial infection” refers to an infection caused by Proteus vulgaris. In one aspect of the invention an “infection” or “bacterial infection” refers to an infection caused by Coagulase-negative Staphylococcus (including Staphylococcus lugdunensis, Staphylococcus capitis, Staphylococcus hominis, and Staphylococcus saprophyticus).

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptococcus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens. In one aspect of the invention an “infection” or “bacterial infection” refers to an infection caused by Enterics. In one aspect of the invention an “infection” or “bacterial infection” refers to an infection caused by Shigella spp. In one aspect of the invention an “infection” or “bacterial infection” refers to an infection caused by Citrobacter.

In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis. In one aspect of the invention “infection” or “bacterial infection” refers to ventilator-associated pneumonia. In one aspect of the invention “infection” or “bacterial infection” refers to intraabdominal infections In one aspect of the invention “infection” or “bacterial infection” refers to gonorrhoeae. In one aspect of the invention “infection” or “bacterial infection” refers to meningitis. In one aspect of the invention “infection” or “bacterial infection” refers to tetanus. In one aspect of the invention “infection” or “bacterial infection” refers to tuberculosis.

In one embodiment, it is expected that the compounds of the present invention will be useful in treating bacterial infections including, but not limited to community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I), or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I), or a pharmaceutically acceptable salt thereof as defined hereinbefore.

According to a further feature of the invention there is provided a method of treating a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococciin a warm-blooded animal, such as a human being, in need of such treatment which comprises administering to said animal an effective amount of a compound of formula (I), or a pharmaceutically acceptable salt thereof as defined hereinbefore.

A further feature of the present invention is a compound of formula (I), and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for use in the production of an anti-bacterial effect in a warm-blooded animal such as a human being.

According to a further aspect of the invention there is provided the use of a compound of formula (I), or a pharmaceutically acceptable salt thereof, in the manufacture of a medicament for use in inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof, in the manufacture of a medicament for use in the treatment of a bacterial infection in a warm-blooded animal such as a human being.

Thus according to a further aspect of the invention there is provided the use of a compound of formula (I), or a pharmaceutically acceptable salt thereof, in the manufacture of a medicament for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

According to a further aspect of the invention there is provided a compound of formula (I), or a pharmaceutically acceptable salt thereof, for use in the production of an anti-bacterial effect in a warm-blooded animal such as a human being.

According to a further aspect of the invention there is provided a compound of formula (I), or a pharmaceutically acceptable salt thereof, for use in inhibition of bacterial DNA gyrase and/or topoisomerase IV in a warm-blooded animal such as a human being.

Thus according to a further aspect of the invention there is provided a compound of formula (I), or a pharmaceutically acceptable salt thereof, for use in the treatment of a bacterial infection in a warm-blooded animal such as a human being.

Thus according to a further aspect of the invention there is provided a compound of formula (I), or a pharmaceutically acceptable salt thereof, for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in a warm-blooded animal such as a human being.

In order to use a compound of the formula (I), or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I), or a pharmaceutically-acceptable salt thereof, and a pharmaceutically-acceptable diluent or carrier.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in inhibition of bacterial DNA gyrase and/or topoisomerase IV in an warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in the treatment of a bacterial infection in a warm-blooded animal, such as a human being.

According to a further aspect of the invention there is provided a pharmaceutical composition which comprises a compound of formula (I), as defined hereinbefore or a pharmaceutically acceptable salt thereof, in association with a pharmaceutically acceptable excipient or carrier for use in the treatment of a bacterial infection selected from community-acquired pneumoniae, hospital-acquired pneumoniae, skin & skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci in an warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:

i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or

ii) anti-infective agents for example, an antifungal triazole e.g. or amphotericin; and/or

iii) biological protein therapeutics for example antibodies, cytokines, bactericidal/permeability-increasing protein (BPI) products; and/or

iv) efflux pump inhibitors.

Therefore, in a further aspect of the invention there is provided a compound of the formula (I), or a pharmaceutically acceptable salt thereof, and a chemotherapeutic agent selected from:

i) one or more additional antibacterial agents; and/or

ii) one or more anti-infective agents; and/or

iii) biological protein therapeutics for example antibodies, cytokines, bactericidal/permeability-increasing protein (BPI) products; and/or

iv) one or more efflux pump inhibitors.

In another embodiment, the invention relates to a method of treating a bacterial infection in an animal, such as a human, comprising administering to the animal an effective amount of a compound of formula (I), or a pharmaceutically acceptable salt thereof, and a chemotherapeutic agent selected from:

i) one or more additional antibacterial agents; and/or

ii) one or more anti-infective agents; and/or

iii) biological protein therapeutics for example antibodies, cytokines, bactericidal/permeability-increasing protein (BPI) products; and/or

iv) one or more efflux pump inhibitors.

As stated above the size of the dose required for the therapeutic or prophylactic treatment of a particular disease state will necessarily be varied depending on the host treated, the route of administration, the severity of the illness being treated, and whether or not an additional chemotherapeutic agent is administered in combination with a compound of the invention. Preferably a daily dose in the range of 1-50 mg/kg is employed. However the daily dose will necessarily be varied depending upon the host treated, the particular route of administration, the severity of the illness being treated, and whether or not an additional chemotherapeutic agent is administered in combination with a compound of the invention. Accordingly the optimum dosage may be determined by the practitioner who is treating any particular patient.

As noted above, one embodiment of the present invention is directed to treating or preventing diseases caused by bacterial infections, wherein the bacteria comprise a GyrB ATPase or topoisomerase IV ATPase enzyme. “Treating a subject with a disease caused by a bacterial infection” includes achieving, partially or substantially, one or more of the following: the reducing or amelioration of the progression, severity and/or duration of the infection, arresting the spread of an infection, ameliorating or improving a clinical symptom or indicator associated with a the infection (such as tissue or serum components), and preventing the reoccurrence of the infection.

As used herein, the terms “preventing a bacterial infection” refer to the reduction in the risk of acquiring the infection, or the reduction or inhibition of the recurrence of the infection. In a preferred embodiment, a compound of the invention is administered as a preventative measure to a patient, preferably a human, before a surgical procedure is preformed on the patient to prevent infection.

As used herein, the term “effective amount” refers to an amount of a compound of this invention for treating or preventing a bacterial infection is an amount which is sufficient to prevent the onset of an infection, reduce or ameliorate the severity, duration, or progression, of an infection, prevent the advancement of an infection, cause the regression of an infection, prevent the recurrence, development, onset or progression of a symptom associated with an infection, or enhance or improve the prophylactic or therapeutic effect(s) of another therapy.

In addition to its use in therapeutic medicine, compounds of formula (I), and their pharmaceutically acceptable salts, are also useful as pharmacological tools in the development and standardization of in-vitro and in-vivo test systems for the evaluation of the effects of inhibitors of DNA gyrase and/or topoisomerase IV in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### Example

The invention is now illustrated but not limited by the following Example in which unless otherwise stated:
(i) evaporations were carried out by rotary evaporation in-vacuo and work-up procedures were carried out after removal of residual solids by filtration;
(ii) operations were generally carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable; the structure of the end-products of the invention were generally confirmed by NMR and mass spectral techniques; proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (6 scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment (FAB) mass spectral data were generally obtained using a Platform spectrometer (supplied by Micromass) run in electrospray and, where appropriate, either positive ion data or negative ion data were collected or using Agilent 1100series LC/MSD equipped with Sedex 75ELSD, run in atmospheric pressure chemical ionisation mode and, where appropriate, either positive ion data or negative ion data were collected; mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported;
(vi) each intermediate was purified to the standard required for the subsequent stage and was characterised in sufficient detail to confirm that the assigned structure was correct; purity was assessed by high pressure liquid chromatography, thin layer chromatography, or NMR and identity was determined by infra-red spectroscopy (IR), mass spectroscopy or NMR spectroscopy as appropriate;
(vii) the following abbreviations may be used:
   ACN is acetonitrile;
   CDCl₃ is deuterated chloroform;
   DBU is 1,8-diazabicyclo[5.4.0]undec-7-ene;
   DCM is dichloromethane;
   DIEA is diisopropyl ethylamine;
   DMF is N,N-dimethylformamide;
   DMSO is dimethylsulfoxide;
   EDC is 1-ethyl-3-(3-dimethyllaminopropyl)carbodiimide;
   EtOAc is ethyl acetate;
   EtOH is ethanol;
   HATU is N-[(dimethylamino)-1H,2,3-triazolo[4,5-b-]pyridin-1-ylmethylene]-N-methylmethanaminium hexafluorophosphate N-oxide;
   HOBT is 1-hydroxybenzotriazole;
   MeOH is methanol;
   MS is mass spectroscopy;
   RT or rt is room temperature;
   SM is starting material;
   TFA is trifluoroacetic acid;
   TFAA is trifluoroacetic anhydride;
   THF is tetrahydrofuran; and
(viii) temperatures are quoted as ° C.

### Example 1

### 1-ethyl-3-(5′-(5-oxo-4,5-dihydro-1,3,4-oxadiazol-2-yl)-4-(pyridin-2-ylethynyl)-3,3′-bipyridin-6-yl)urea

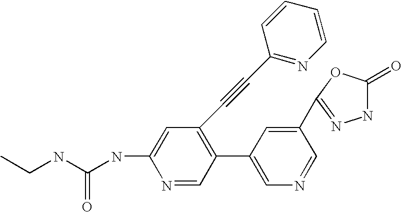

A mixture of (1-(4-bromo-5′-(5-oxo-4,5-dihydro-1,3,4-oxadiazol-2-yl)-3,3′-bipyridin-6-yl)-3-ethylurea (Intermediate 1, 62 mg, 0.15 mmol), 2-ethynylpyridine (15.78 mg, 0.15 mmol), copper(I) iodide (1.457 mg, 7.65 μmol), triethylamine (0.064 mL, 0.46 mmol), and dichlorobis(triphenylphosphine)palladium(II) (5.37 mg, 7.65 μmol) was suspended in acetonitrile (5 mL) and heated for 6 hours in a microwave. The reaction mixture was cooled to room temperature, filtered through celite and the filtrate was concentrated under reduced pressure. Purification by column chromatography (silica, eluted with Hex/EtOAc) to give the desired product (28 mg).

MS (ESP): 428 (MH⁺) for C₂₂H₁₇N₇O₃.

¹H-NMR (DMSO-d₆) δ: 1.11(t, 3H); 3.21 (q, 2H); 7.43 (t, 1H); 7.52 (d, 1H); 7.62 (t, 1H); 7.83 (t, 1H); 7.88 (s, 1H); 8.47 (s, 1H); 8.51 (s, 1H); 8.59 (d, 1H); 9.0 (s, 2H); 9.49 (s, 1H); 12.80 (br, 1H).

### Example 2

### 1-Ethyl-3-(4-ethynyl-5′-(5-oxo-4,5-dihydro-1,3,4-oxadiazol-2-yl)-3,3′-bipyridin-6yl)urea

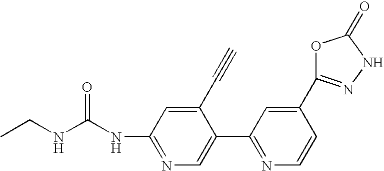

1-Ethyl-3-(5′-(5-oxo-4,5-dihydro-1,3,4-oxadiazol-2-yl)-4-((trimethylsilyl)ethynyl)-3,3′-bipyridin-6-yl)urea (Example 3, 84 mg, 0.20 mmol) was suspended in methanol (5 ml). NaOH (2 ml, 2.00 mmol) was added and the mixture was stirred at room temperature for 2 hrs. Aqueous HCl solution(2N) was added until the pH reached 6.5. DCM(10 ml) was added and the organic layer was separated, washed with brine and dried over MgSO₄, then filtered and concentrated to a volume of 2 mL. Hexanses were added and the resulting precipitate was filtered and washed with DCM, collected as the desired product (25 mg).

MS (ESP) 351 (MH⁺) for C₁₇H₁₄N₆O₃

¹H-NMR (DMSO-d₆): 1.10 (t, 3H); 3.20 (m, 2H); 4.66 (s, 1H); 7.61 (m, 1H); 7.78 (s, 1H); 8.34 (m, 1H); 8.41 (s, 1H); 8.92 (d, 1H); 8.98 (d, 1H); 9.43 (s, 1H); 12.84 (br, 1H) ppm

### Example 3

### 1-Ethyl-3-(5′-(5-oxo-4,5-dihydro-1,3,4-oxadiazol-2-yl)-4-((trimethylsilyl)ethynyl)-3,3′-bipyridin-6-yl) urea

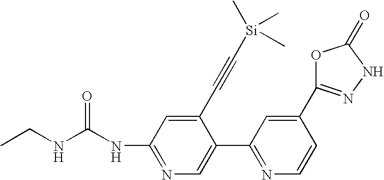

1-(4-Bromo-5′-(5-oxo-4,5-dihydro-1,3,4-oxadiazol-2-yl)-3,3′-bipyridin-6-yl)-3-ethylurea (Intermediate 1, 400 mg, 0.99 mmol), ethynyltrimethylsilane (116 mg, 1.18 mmol), copper(I) iodide (18.80 mg, 0.10 mmol), Et₃N (0.550 mL, 3.95 mmol), and Pd(PPh₃)4 (57.1 mg, 0.05 mmol) were combined in anhydrouse DMF (10 mL) and heated at 80° C. for 4 hours. After cooling down to room temperature, the crude sample was filtered through celite and the filtrate was concentrated and purified by column chromatography on silica gel(Hex/EtOAc) to give the tile compound (160 mg).

MS (ESP) 423 (MH⁺) for C₂₀H₂₂N₆O₃Si

¹H-NMR (DMSO-d₆): 0.12 (s, 9H); 1.10 (t, 3H); 3.20 (m, 2H); 7.57 (m, 1H); 7.72 (s, 1H); 8.41 (m, 1H); 8.45 (s, 1H); 8.92 (d, 1H); 8.99 (d, 1H); 9.41 (s, 1H); 12.86 (s, 1H) ppm

Intermediate 1

### 1-(4-bromo-5′-(5-oxo-4,5-dihydro-1,3,4-oxadiazol-2-yl)-3,3′-bipyridin-6-yl)-3-ethylurea

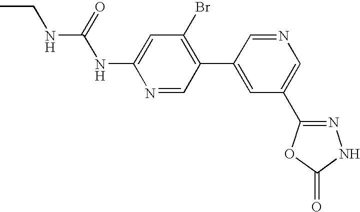

A mixture of (1-(4-bromo-5′-(hydrazinecarbonyl)-3,3′-bipyridin-6-yl)-3-ethylurea (Intermediate 2, 60 mg, 0.16 mmol), 1,1′-carbonylbis(1H-imidazole) (34.4 mg, 0.21 mmol) and diisopropylethylamine (0.041 ml, 0.24 mmol) in DMF (3 ml) was heated at 50° C. for 4 hours and then cooled down to room temperature. The crude residue was concentrated under reduced pressure and purified by column chromatography (silica, 5% methanol in dichloromathane) to give the desired product as a solid (62 mg).

MS (ESP): 407 (MH⁺) for C₁₅H₁₃BrN₆O₃.

¹H-NMR (DMSO-d₆) δ: 1.09 (t, 3H); 3.19 (t, 2H); 7.48 (t, 1H); 8.04 (s, 1H); 8.23 (t, 1H); 8.29 (s, 1H); 8.80 (d, 1H); 9.0 (d, 1H); 9.45 (s, 1H).

Intermediate 2

### 1-(4-bromo-5′-(hydrazinecarbonyl)-3,3′-bipyridin-6-yl)-3-ethylurea

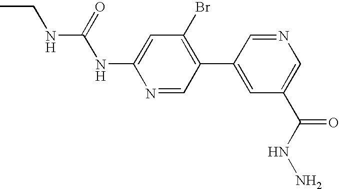

Ethyl 4′-bromo-6′-(3-ethylureido)-3,3′-bipyridine-5-carboxylate (Intermediate 3, 1.32 g, 2.85 mmol) and hydrazine hydrate (1.416 ml, 28.53 mmol) were mixed in ethanol (20 ml), heated at 80° C. for 2 d, and then cooled down to room temperature. The resulting residue was diluted with ethyl acetate. The resulting precipitate was collected by filtration and washed with ethyl acetate (920 mg).

MS (ESP): 381 (MH⁺) for C₁₄H₁₅BrN₆O₂

¹H-NMR (DMSO-d₆): 1.08 (t, 3H); 3.17 (q, 2H); 3.58 (br, 2H); 7.43 (t, 1H); 8.05 (s, 1H); 8.27 (s, 2H); 8.85 (s, 1H); 9.03 (s, 1H); 9.43 (s, 1H); 11.15 (br, 1H).

Intermediate 3

### ethyl 4′-bromo-6′-(3-ethylureido)-3,3′-bipyridine-5-carboxylate

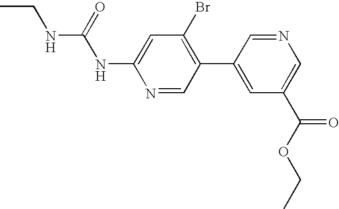

A mixture of 1-(4-bromo-5-iodopyridin-2-yl)-3-ethylurea (Intermediate 4, 1.33 g, 3.59 mmol), ethyl 5-(4,4,5,5-tetramethyl-1,3,2-dioxaborolan-2-yl)nicotinate (1.049 g, 3.59 mmol), paladium-tetrakistriphenylphosphine (0.415 g, 0.36 mmol) and K₂CO₃ (0.745 g, 5.39 mmol) was suspended in a mixture of DMF (10 mL) and water (1.000 mL). The suspension was degassed and purged with nitrogen and heated at 100° C. for 1.5 h. The reaction mixture was cooled to room temperature and filtered and the filtrate was concentrated under reduced pressure. Purification by column chromatography on silica gel gave the desired product (1.32 g).

MS (ESP): 395 (MH⁺) for C₁₆H₁₇BrN₄O₃.

¹H-NMR (CDCl₃): 1.29 (t, 3H); 1.45 (t, 3H); 3.45 (q, 2H); 4.47 (q, 2H); 7.30 (br, 1H); 8.12 (s, 1H); 8.38 (t, 1H); 8.84 (2s, 2xH); 9.29 (s, 1H).

Intermediate 4

### 1-(4-bromo-5-iodopyridin-2-yl)-3-ethylurea

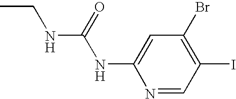

A solution of 4-bromo-5-iodopyridin-2-amine (Intermediate 5, 3.2g, 10.71 mmol) in dry chloroform (15 mL), was treated with isocyanatoethane (2.52 mL, 32.12 mmol) and the reaction mixture was heated to reflux for 24 h. The reaction mixture was cooled to room temperature and hexane was added. The desired product was formed a precipitate which was collected by filtration (yield: 3.14 g).

MS (ESP⁺): 371 (MH⁺) for C₈H₉BrIN₃O.

¹H-NMR (DMSO-d₆): 1.06 (t, 3H); 3.32 (q, 2H); 7.24 (br, 1H); 8.05 (s, 1H); 8.52 (s, 1H); 9.31 (s, 1H).

Intermediate 5

### 4-bromo-5-iodopyridin-2-amine

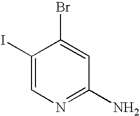

To a solution of 4-bromopyridin-2-amine (2.5 g, 14.45 mmol) in DMF (6 mL)/chloroform (20 mL), 1-iodopyrrolidine-2,5-dione (6.50 g, 28.90 mmol) was added. The reaction mixture was stirred at 45° C. for 2 d. The chloroform was removed under reduced pressure and the remaining solution was poured into water (15 mL) and extracted with EtOAc (15 mL×3). The organic phase was concentrated under reduced pressure. Purification by column chromatography (silica, eluted with Hex/EtOAc) provided the title compound (3.2 g).

MS (ESP): 298(MH⁺) for C₅H₄BrIN₂.

¹H-NMR (DMSO-d₆): 4.51 (br, 2H); 6.80 (s, 1H); 8.35 (s, 1H).

1. A compound of formula (I): 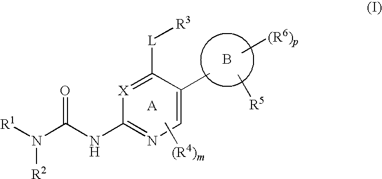
or a pharmaceutically acceptable salt thereof, wherein:
X is CH;
L is —C≡C—;
Ring B is pyridinyl;
R¹ is C₁₋₄alkyl;
R² is hydrogen;
R³ is a hydrogen;
R⁵ is selected from the group consisting of 5-hydroxy-1,3,4-oxadiazol-2-yl;
m is 0; and
p is 0.

2. A pharmaceutical composition comprising a compound of claims 1, or a pharmaceutically acceptable salt thereof, and a pharmaceutically acceptable excipient or carrier.

3. A method of treating a bacterial infection in a warm-blooded animal in need thereof, comprising administering to the animal an effective amount of a compound of claims 1, or a pharmaceutically acceptable salt thereof.

4. The method of claim 3, wherein the warm-blooded animal is a human.

5. The method of claim 3, wherein the bacterial infection is community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.

6. A compound of formula (I): 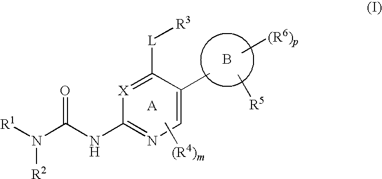
or a pharmaceutically acceptable salt thereof, wherein:
X is CH;
L is —C≡C—;
Ring B is pyridinyl;
R¹ is C₁₋₄alkyl;
R² is hydrogen;
R³ is a trimethylsilyl;
R⁵ is selected from the group consisting of 5-hydroxy-1,3,4-oxadiazol-2-yl;
m is 0; and
p is 0.

7. A pharmaceutical composition comprising a compound of claims 6, or a pharmaceutically acceptable salt thereof, and a pharmaceutically acceptable excipient or carrier.

8. A method of treating a bacterial infection in a warm-blooded animal in need thereof, comprising administering to the animal an effective amount of a compound of claims 6, or a pharmaceutically acceptable salt thereof.

9. The method of claim 8, wherein the warm-blooded animal is a human.

10. The method of claim 8, wherein the bacterial infection is community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.

11. A compound of formula (I): 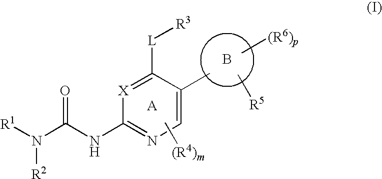
or a pharmaceutically acceptable salt thereof, wherein:
X is CH;
L is —C≡C—;
Ring B is pyridinyl;
R¹ is C₁₋₄alkyl;
R² is hydrogen;
R³ is a pyridinyl;
R⁵ is selected from the group consisting of 5-hydroxy-1,3,4-oxadiazol-2-yl;
m is 0; and
p is 0.

12. A pharmaceutical composition comprising a compound of claims 11, or a pharmaceutically acceptable salt thereof, and a pharmaceutically acceptable excipient or carrier.

13. A method of treating a bacterial infection in a warm-blooded animal in need thereof, comprising administering to the animal an effective amount of a compound of claims 11, or a pharmaceutically acceptable salt thereof.

14. The method of claim 13, wherein the warm-blooded animal is a human.

15. The method of claim 13, wherein the bacterial infection is community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections, Penicillin-resistant Streptococcus pneumoniae, methicillin-resistant Staphylococcus aureus, methicillin-resistant Staphylococcus epidermidis or Vancomycin-Resistant Enterococci.
